(19) United States
(12) Patent Application Publication
(10) Pub. No.: US 2024/0252570 A1
(43) Pub. Date: Aug. 1, 2024

(54) EXTRACTION AND PURIFICATION OF THE DISTILLATION RESIDUES OF BLACK SPRUCE AND LABRADORS TEA AND COMBINATION THEREOF

(71) Applicant: SILICYCLE INC., Québec (CA)

(72) Inventors: Alain THIBODEAU, Québec (CA); François BELAND, L'Ancienne-Lorette (CA); Meryem BOUCHOUCHA, Québec (CA); Ludivine CHAURAND, Québec (CA); Maëlle DERRIEN, Québec (CA)

(21) Appl. No.: 18/560,528

(22) PCT Filed: May 19, 2022
(86) PCT No.: PCT/CA2022/050790

§ 371 Date: Nov. 13, 2023

Related U.S. Application Data
(60) Provisional application No. 63/192,928, filed on May 25, 2021.

(51) Int. Cl. A61K 36/15; A61K 36/45; A61P 39/06

(52) U.S. Cl. CPC A61K 36/15; A61K 36/45; A61P 39/06

(57) ABSTRACT

It is provided an antioxidant composition comprising a black spruce bark extract and a Labrador's tea aerial part residue extract and method of producing the extracts. The composition as described herein is rich in polyphenolic content and is used for protecting and/or treating a subject against oxidative stress or a subject cells against oxidative stress.

### CROSS-REFERENCE TO RELATED APPLICATIONS

The present application claims benefit of U.S. Provisional Application No. 63/192,928 filed May 25, 2021, the content of which is hereby incorporated by reference in its entirety.

### TECHNICAL FIELD

It is described an antioxidant composition comprising a black spruce bark extract and a Labrador's tea aerial part residue extract.

### BACKGROUND

Agricultural and industrial residue valorization has become an important parameter in the fields of food, nutraceutical, cosmetics and transformation industries. Providing a “second life” to otherwise discarded residues has gained interest for economical and ecological reasons. Furthermore, the availability of finished products made of, or containing, such valorized residues becomes a motivation for customers when it comes to make a purchase decision. Several studies have demonstrated the high valorization potential of agriculture and logging-derived by-products. Indeed, numerous unused residues from these activities still contains a large variety of bio-active molecules. Among those, phytochemicals, particularly the members of the wide polyphenol group, could in turn be transformed into value-added ingredients.

For instance, studies have been performed to explore the potential of by-products generated from essential oil preparation. In this process, various vegetable substrates are submitted to hot water distillation. The steam water extracts the essential oil separating it from the vegetable substrate. Following the extraction process, the vegetable substrate is usually discarded. Studies have shown that post-distillation vegetable substrates still contain high concentrations of polyphenolic compounds and could be used as economic and environmental-friendly sources. For example, the valorization of the distillation residues from Laurus nobilis, Alpinia zerumbet and rose oil distillation water successfully produced extracts exhibiting high concentration in polyphenolic compounds. Previous studies have also been performed for the valorization of black spruce water-distillation residues with positive results.

Labrador's tea is a widely used medicinal plant among First Nations population in Canada. This plant was used for the treatment of several chronic diseases such as inflammatory conditions, respiratory illnesses, asthma, rheumatisms and liver pathologies. These effects are in correlation with the anti-oxidant, anti-inflammatory and anti-cancer activities of this plant described by several authors. The following compounds were identified in Labrador's tea: (+) catechin, chlorogenic acid, (−) epicatechin, procyanidin B2, procyanidin A1, and several quercetin glycosides (Eid et al., 2016, Journal of Ethnopharmacology, 178: 251-257).

Another example of a by-product valorization is the extraction of tree bark. In addition to lumber, forestry and logging activities generate enormous amounts of bark. Bark residues can be used as complements for fertilizers or as a substrate to generate energy. Studies have shown that bark residues are endowed with bio-active molecules generally part of the global polyphenol group. Black spruce (Picea mariana) is a coniferous very popular for lumber preparation. Therefore, black spruce tree bark is an abundant source of biomass that could be used as a substrate for the extraction of polyphenolic compounds.

Picea mariana has been used as a folk remedy for skin and soft tissues condition and as analgesic by Canada's First Nations. This plant is known to possess several beneficial health effects related to its phytochemical content. Indeed, black spruce bark is known to possess high concentrations of polyphenols, in particular dihydroquercetin and proanthocyanidin, extensively studied for their implications in the prevalence reduction of several chronic diseases. Black spruce bark hot water extracts have been previously studied and exhibits an interesting composition in low molecular weight phenolic compounds mainly belonging to the stilbene family such trans-resveratrol, trans-isorhapontin, trans-piceide and trans-astringin (Francezon & Stevanovic, 2017, Industrial Crops ad Products, 108: 348-354).

Polyphenols and related molecules are known for various biological activities especially antioxidant actions. With regards to the antioxidant action, a combination of polyphenols can be more potent than individual molecules. This can be explained by a more efficient chelation and inactivation of free radicals responsible for a pro-oxidant reaction.

There is still a need to be provided with valorization products from natural sources like the Labrador tea plant and tree bark.

### SUMMARY

It is provided a composition comprising a black spruce bark extract and a Labrador's tea aerial part residue extract.

In an embodiment, the composition comprises a ratio from about 1 black spruce bark extract for 9 Labrador's tea aerial part residue extract (w/w) to about 9 black spruce bark extract for 1 Labrador's tea aerial part residue extract (w/w).

In another embodiment, the composition comprises 50% (w/w) of the black spruce bark extract and 50% (w/w) of the Labrador's tea aerial part residue extract.

In a further embodiment, the black spruce bark extract is from untreated bark harvested from logging activities.

In another embodiment, the black spruce bark extract is from distillation residues.

In an embodiment, the Labrador's tea aerial part extract is from distillation residues.

In a supplemental embodiment, the Labrador's tea aerial part extract is from untreated harvested leaves.

In an embodiment, the black spruce bark extract has a polyphenolic content ranging from 10% to 40% (w/w).

In another embodiment, the Labrador's tea aerial part extract has a polyphenolic content ranging from 20% to 50% (w/w).

In a further embodiment, the composition is in a liquid form, an oil or in a powder form.

In a further embodiment, the composition is an antioxidant composition.

In an alternate embodiment, the composition protects of treat a subject against oxidative stress.

In a further embodiment, the subject is a human or an animal.

In another embodiment, the composition protects or treat a subject cells against oxidative stress.

In an embodiment, the cells are isolated cells.

It is further provided the use of the composition as described herein for protecting and/or treating a subject against oxidative stress.

It is also provided the use of the composition as described herein for protecting and/or treating ex vivo a subject cells against oxidative stress.

It is additionally provided a method for protecting and/or treating a subject against oxidative stress comprising administering an effective amount of the composition as described herein to a subject.

In an embodiment, the composition is administered to isolated cells of said subject.

### BRIEF DESCRIPTION OF THE DRAWINGS

Reference will now be made to the accompanying drawings.

FIG. 1 illustrates a diagram flowchart of the extraction and purification of black spruce bark in accordance to an embodiment.

FIG. 2 illustrates a diagram flowchart of the extraction and purification of Labrador's tea aerial parts as encompassed in an embodiment.

### DETAILED DESCRIPTION

It is provided an antioxidant composition comprising a black spruce bark extract and a Labrador's tea aerial part residue extract.

The present disclosure provides for a process for the valorization of water distillation residues from Labrador's tea (Rhododendron groenlandicum) aerial parts and black spruce (Picea mariana) bark obtained from logging activity. It is described a process using a solvent such as hot water, ethanol or a combination of both producing several extracts with different concentrations of total polyphenols from these residues. As described herein, a final product consisting of the combination of the said Labrador tea and black spruce extracts is provided. The phytochemical characterization of this extract combination (Boreaplex™) exhibits a rich polyphenolic profile and possesses strong antioxidant activities demonstrated with a human cell-based assay. In fact, the antioxidant properties of Boreaplex™ is superior to that of each individual Labrador tea and black spruce extract. Boreaplex™ can be used as an ingredient in nutraceuticals and cosmeceuticals formulation.

As encompassed herein, Boreaplex™ consists of the combination of crude and purified extracts to obtained:

| Boreaplex ™ | Labrador's tea aerial part | Black spruce bark crude |
|---|---|---|
| | residue crude aqueous | aqueous extract (50%) or |
| | extract (50%) or | Black spruce bark |
| | Labrador's tea aerial part | purified extract (50%) |
| | residue purified extract | |
| | (50%) | |

Accordingly, it is described a solvent extraction, such e.g. water, ethanol or a combination thereof, and purification process using black spruce bark and Labrador's tea aerial part distillation residues. It results in extracts consisting of different concentrations as well as chromatographic profile in polyphenols. A final product, called Boreaplex™, is provided consisting of a combination of the obtained extracts from black spruce bark and Labrador tea aerial part distillation residues. These extracts are combined in different ratios according to the desired concentration of phenolic constituents. The combination of these extracts provides a composition enriched in different members of the polyphenol family not initially present in one or other extracts obtained from black spruce bark or Labrador's tea aerial part residues. Polyphenols have powerful anti-oxidant activities and have been described as anti-cancer, hepatoprotective, chemoprotective among others.

Distillation residues are usually destined to waste or have a limited used as fertilizers or in animal feed. However, these residues still contain high concentration of polyphenol and other phytomolecules which are not extracted under the distillation conditions used for the production of essential oils. The distillation process used for instance is for example the water or steam distillation. The distillation process provides positive physical modifications of the vegetable substrate enabling a more efficient mass transfer for the extraction of polyphenols and other extractables. Once hydro or steam-distillation is completed and essential oils are collected, the distillation residue is dried and grinded. The bark obtained from black spruce trees is also dried and grinded. Both vegetable substrates are added separately to extraction reactors containing water or ethanol as solvents. The vegetable substrates and the solvent are heated at a temperature between 30° C. and 100° C. and the aqueous extraction takes place for a period varying between 0.25 hour and 3 hours. The extract so obtained is filtered and can be dried directly to obtain an aqueous crude extract. The extract can be further purified through a resin after the filtration in order to eliminate residual sugars present in the extract and to concentrate the polyphenolic fraction. The crude or purified extracts are then concentrated and dried by spray drying or lyophilization. These extracts are then combined to obtain the Boreaplex™ final product.

It is provided a method for the extraction and purification of black spruce bark and Labrador's tea aerial part residues. The extracts encompassed herein are highly concentrated in polyphenols. Once combined, the final product named Boreaplex™ is composed with different members of polyphenolic family molecules found in both black spruce bark and Labrador's tea aerial part extracts. Moreover, Boreaplex™ exhibits the highest anti-oxidant capacity compared to individual extract.

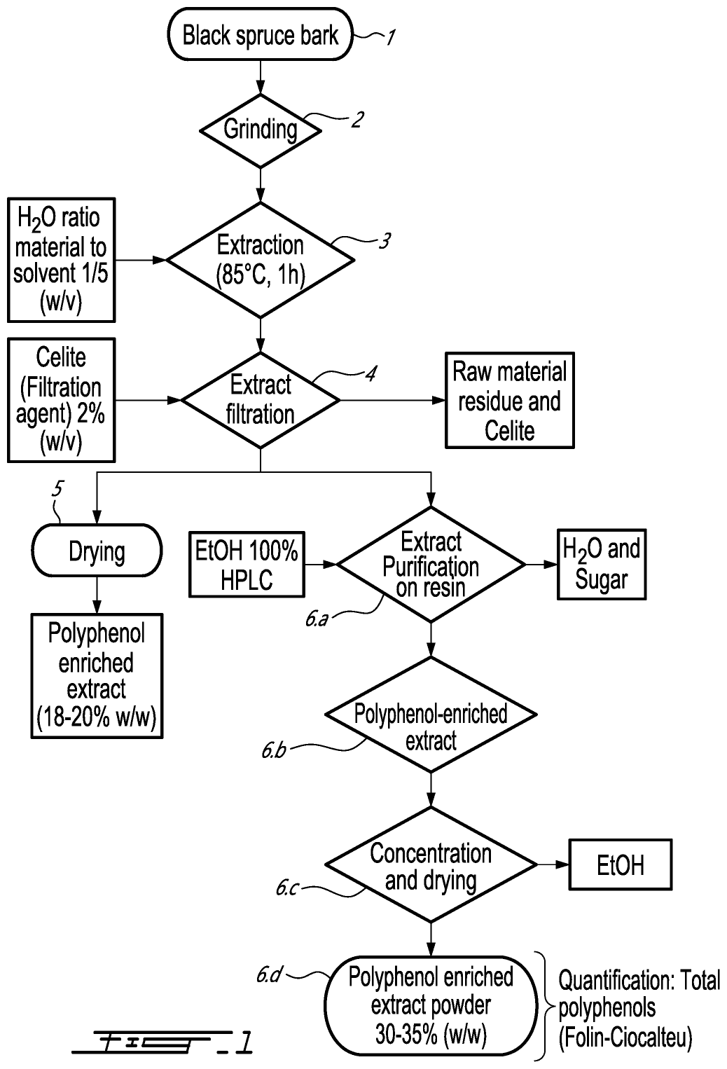

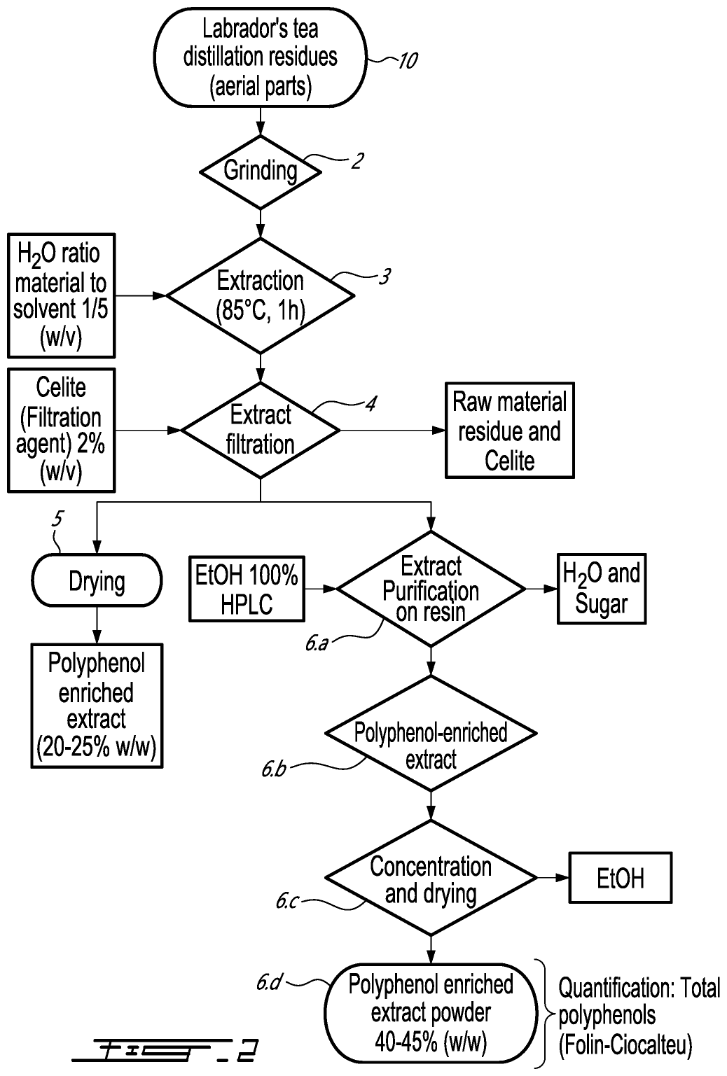

In one embodiment, as seen in FIGS. 1 and 2, the black spruce bark residues are obtained from logging activities (1). Alternatively, it is encompassed that the black spruce bark are obtained from water or steam distillation.

Similarly, the Labrador's tea aerial part residues are obtained from water or steam distillation (10, FIG. 2) or from harvesting activities.

In one embodiment, the distillation residue is pre-treated before the extraction. The pretreatment consists in freezing the distillation residues in order to ensure an optimal conservation of the raw material until the day of processing. On the day of the processing, the pretreatment consists of grinding 2 the frozen raw material. Grinding enhances the contact surface between material and solvent, favoring the extraction of phytochemicals.

In one embodiment, the raw material is extracted 3 with hot water for 1h and at a temperature comprised from 30 to 100° C.

In one embodiment, the ratio solvent to raw material is comprised between 1-3 to 1-10.

In one embodiment, the extract is filtrated 4 using an appropriate technique (centrifugation, membrane separation or ultrafiltration) to separate it from the non-soluble fraction. A further filtration step is needed if the extract is purified on resin. The addition of a filtrating agent, using Celite™ or other appropriate matrices, at a concentration of 2% (filtering agent to volume solution; w/v) help preventing any clogging issues at this step.

In a further embodiment, the aqueous crude extract of black spruce bark residue and of Labrador's tea aerial part residue is concentrated and dried 5 by lyophilization or spray-drying to obtain an extract enriched in total polyphenol (18-20%, w/w and 20-25%, w/w respectively).

In one embodiment, the black spruce bark aqueous crude extract and Labrador's tea aerial part residue aqueous crude extract are purified separately onto a resin. The purification step ensures the removal of sugars known to cause stickiness of the final powder sometimes associated with hygroscopicity. The low glass transition of sugars and other organic acids are responsible for these issues and their removal help for the production of a quality powder extract at the end of the process. The removal of sugars as well as other non-retained compounds ensure the concentration of the polyphenolic fraction in the final extract.

In one embodiment, the extract obtained is then purified using a resin 6a for the removal of sugars and to concentrate the total polyphenol in a final extract. As encompassed herein, the resin consists e.g. in a non-ionic, hydrophobic polymeric adsorbent. Its adsorptive properties derived from its macroporous structure, high surface area and the aromatic nature of its surface.

In one embodiment, the pretreatment of the column consists in washing the possible salt used to inhibit bacterial growth. Prior to the first utilization, the column was also activated by water miscible organics solvents. The chosen solvent is then displaced with water prior to utilization of the column.

In one embodiment, the crude aqueous extract (black spruce bark or Labrador's tea aerial part distillation residues) is loaded onto the resin and the polyphenolic fraction is retained 6b onto it.

In an embodiment, the resin is washed with several column volumes of water to remove all of the non-phenolic compounds especially sugars. This step is monitored through a Brix measurement of the solution.

In an embodiment, the polyphenolic fraction remaining onto the resin is eluted with 1 to 5 column volumes of 100% HPLC grade water soluble organic solvent, e.g ethanol, methanol, acetone or isopropanol.

In an embodiment, the eluted extract is concentrated and dried 6c using an appropriate technique (spray drying, evaporation or lyophilization) to obtain a high-quality extract powder.

The Labrador tea aerial part residue and black spruce bark purified extract obtained 6d contain respectively 40 to 45% and 30 to 35% total polyphenolic content. Total polyphenols is determined e.g. by the Folin-Ciocalteu method.

In one embodiment, the crude and/or purified extract are homogeneously mixed by any means to produce a combined extract (Boreaplex™). Boreaplex™ can be composed with a ratio of 50/50 (w/w) black spruce bark purified extract/Labradors tea purified extract obtained by the developed methodology from steam or water distillation residues. Boreaplex™ can be composed of 50/50 (w/w) black spruce bark crude extract/Labradors tea crude extract obtain by the developed methodology from steam or water distillation residues.

In an embodiment, the composition described herein is in a liquid form, an oil or a powder form.

In one embodiment, individual extracts of black spruce bark and Labrador's tea aerial part residue as well as the combined product named Boreaplex™ exhibit strong anti-oxidant activities. These effects were showed using a cell-based anti-oxidant assay with the human skin fibroblast WS1 cell line. Boreaplex™ exhibits the lowest IC₅₀, meaning it possesses higher anti-oxidant activity than the Labrador's tea aerial part residue and black spruce bark individual extracts (see Example V).

Pro-oxidant stress has been described as resulting from a number of different oxidative challenges, including e.g. radiation, metabolism of environmental pollutants and administered drugs, as well as immune system response to disease or infection. In controlled situations, oxidative materials are generated in order to attack invading organisms.

An oxidative stress is known as the reactions of partially reduced oxygen products and radical and non-radical species derived from them. A variety of reactive nitrogen species derived from the reactions of nitric oxide also play important roles in oxidative stress.

Oxidative stress has been implicated in human and animal diseases causing cell damage. Intake of anti-oxidant in humans has been reported to decrease the risk of developing specific forms of cancer and to enhance immune function. Oxidative stress, is also known to directly contribute to cataract formation, osteoarthritis, aging processes, degenerative diseases and cancer.

Accordingly, the composition described herein can be used as an anti-oxidant composition for protecting and treating a subject, including humans and animals, against oxidative stress. It is further encompassed that the composition described herein used as an anti-oxidant composition for protecting and treating cells against oxidative stress.

### Example I

### Labrador's Tea Aerial Part Residue Aqueous Crude Extract Preparation

Labrador's tea aerial part residues from steam distillation were frozen until the extraction process occurred. The day of the process, 40.17 g of residues were grinded and contacted with H₂O, with a volume corresponding to 5 times the mass of the residues. The extraction occurred at 85° C. for 1h and the crude extract was then added with a filtering agent to enhance the filtration step efficiency. The filtering agent was Celite™ and the ratio added was 2% (w/v). The filtration was performed under vacuum. The drying of the crude extract was performed by lyophilization. The obtained extract had a total polyphenol concentration of 23.6%. The quantification of total polyphenol was performed using the Folin-Ciocalteu method.

### Example II

### Labrador's Tea Aerial Part Residue Crude Extract Purification

Labrador's tea aerial part residues from steam distillation were frozen until the extraction process occurred. On the day of the process, 61.5 g of residues were grinded and contacted with H₂O, with a volume corresponding to 5 times the mass of the residues. The extraction occurred at 85° C. for 1h and the crude extract was then added with a filtering agent to enhance the filtration step efficiency. The filtering agent was Celite™ and the ratio added was 2% (w/v). The filtration was performed under vacuum. The drying of the crude extract was performed by lyophilization. The obtained extract had a total polyphenol concentration of 24.3%. The quantification of total polyphenol was performed using Folin-Ciocalteu methodology. The crude extract was then contacted with 70 g of activated Amberlite XAD16N polymeric resin (Amberlite® XAD16N 20-60 mesh; CAS Number: 104219-63-8; Sigma Aldrich) for the fractionation of the total polyphenols. The resin was washed several times with water to ensure the removal of non-polyphenolic compounds. The polyphenolic fraction was then eluted with one column volume of HPLC grade ethanol. The extract was then concentrated by rotary evaporation until dryness. The obtain extract had a concentration of total polyphenol of 44.2%.

### Example III

### Black Spruce Bark Crude Extract Preparation

Black spruce bark pieces were frozen until the extraction process occurred. On the day of the process, 40.17 g of the material were grinded and contacted with H₂O, with a volume corresponding to 5 times the mass of the residues. The extraction occurred at 85° C. for 1h and the crude extract was then added with a filtering agent to enhance the filtration step efficiency. The filtering agent was Celite™ and the ratio added was 2% (w/v). The filtration was performed under vacuum. The drying of the crude extract was performed by lyophilization. The obtained extract had a total polyphenol concentration of 16.6%. The quantification of total polyphenol was performed using Folin-Ciocalteu method.

### Example IV

### Black Spruce Bark Residue Crude Extract Purification

Black spruce bark pieces were frozen until the extraction process occurred. The day of the process, 61.5 g of residues were grinded and contacted with H₂O, with a volume corresponding to 5 times the mass of the residues. The extraction occurred at 85° C. for 1h and the crude extract was then added with a filtrating agent to enhance the filtration step efficiency. The filtrating agent was Celite™ and the ratio added was 2% (w/v). The filtration was performed under vacuum. The drying of the crude extract was performed by lyophilization. The obtained extract had a total polyphenol concentration of 15.2% (Table 1). The quantification of total polyphenol was performed using Folin-Ciocalteu methodology. The crude extract was then contacted with 70 g of activated Amberlite XAD16N polymeric resin (Amberlite® XAD16N 20-60 mesh; CAS Number: 104219-63-8; Sigma Aldrich) for the fractionation of the total polyphenols. The resin was washed several times with water to ensure the removal of non-polyphenolic compounds, in particularly sugars. The polyphenolic fraction was then eluted with one column volume of HPLC grade ethanol. The obtain extract had a concentration of total polyphenol of 33%.

TABLE 1
Total polyphenol content of extracts

| | %(w/w) Total Polyphenols |
|---|---|
| Extract | (Gallic acid Eq) |
| Black spruce bark aqueous crude extract | 15.2 |
| Black spruce bark purified extract (resin) | 33.0 |
| Labrador's tea aerial part residue aqueous | 24.3 |
| crude extract | |
| Labrador's tea aerial part residue purified | 44.2 |
| extract (resin) | |

### Example V

### Cell-Based Assay Using DCFH Oxidation for the Determination of Antioxidant Properties

The anti-oxidant activity of the studied extracts was assessed using a human cell-based assay. Cells were washed with 150 ml of pH 7.4 phosphate buffer saline (PBS) and incubated for 30 min with 100 ml Hank's buffered salt solution (pH 7.4) containing 5 mM 2′, 7′-dichlorofluorescin-diacetate (DCFH-DA). The cells were then washed again with 150 ml PBS. To assess antioxidant activity, the cells were incubated for 1 h with increasing concentrations of the Labrador's tea aerial part residue extract, black spruce bark extract or a combination of both as Boreaplex™ in the absence or presence of 200 mM tert-buthylhydroperoxide (t-BuOOH). Fluorescence was measured immediately after t-BuOOH administration and also 90 min later on the automated plate reader using an excitation wavelength of 485 nm and an emission wavelength of 530 nm. Antioxidant activity is expressed as 2′, 7′-dichlorofluorescin-diacetate (DCFH-DA)IC₅₀, meaning the concentration of extract enable the inhibition of DCFH oxidation induced by t-BuOOH by 50%. (Girard-Lalancette et al., 2009, Food Chemistry, 115: 720-726) (see Table 2).

TABLE 2
Antioxidant activity comparison

| Extracts | IC₅₀ | |
|---|---|---|
| Black spruce bark crude extract 100% water | 1.3 ± 0.2 | μg/ml |
| Labrador's tea aerial part residue crude extract | 0.9 ± 0.1 | μg/ml |
| 100% water | | |
| Boreaplex ™ (50/50; w/w Labrador's tea and black | 0.62 ± 0.06 | μg/ml |
| spruce crude extract) | | |

The combination of the Labrador's tea aerial part distillation residue extract with the black spruce bark extract shows a significant higher anti-oxidant activity (IC₅₀ 0.62±0.06 μg/ml) when compared to the individual Labrador's tea aerial part residue (IC₅₀ 0.9±0.1 μg/ml; P<0.02) and black spruce bark (IC₅₀ 1.3±0.2 μg/ml; P<0.005) extracts. These results support the rationale to combine Labrador's tea aerial distillation residues with black spruce water crude extract in the form of the product Boreaplex™ to significantly increase the intracellular anti-oxidant activity.

While the present description has been described in connection with specific embodiments thereof, it will be understood that it is capable of further modifications and this application is intended to cover any variations, uses, or adaptations and including such departures from the present disclosure as come within known or customary practice within the art to and as may be applied to the essential features hereinbefore set forth, and as follows in the scope of the appended claims.

What is claimed is:

1. A composition comprising a black spruce bark extract and a Labrador's tea aerial part residue extract.

2. The composition of claim 1, comprising a ratio from about 1 black spruce bark extract/9 Labrador's tea aerial part residue extract (w/w) to about 9 black spruce bark extract/1 Labrador's tea aerial part residue extract (w/w).

3. The composition of claim 1, comprising 50% (w/w) of the black spruce bark extract and 50% (w/w) of the Labrador's tea aerial part residue extract.

4. The composition of claim 1, wherein the black spruce bark extract is from untreated bark harvested from logging activities.

5. The composition of claim 1, wherein the black spruce bark extract is from distillation residues.

6. The composition of claim 1, wherein the Labrador's tea aerial part extract is from distillation residues.

7. The composition of claim 1, wherein the Labrador's tea aerial part extract is from untreated harvested leaves.

8. The composition of claim 1, wherein the black spruce bark extract has a polyphenolic content ranging from 10% to 40% (w/w).

9. The composition of claim 1, wherein the Labrador's tea aerial part extract has a polyphenolic content ranging from 20% to 50% (w/w).

10. The composition of claim 1, wherein the composition is in a liquid form, an oil or in a powder form.

11. The composition of claim 1, wherein said composition is an antioxidant composition.

12. The composition of claim 1, wherein said composition protects or treat a subject against oxidative stress.

13. The composition of claim 12, wherein said subject is a human or an animal.

14. The composition of claim 12, wherein said composition protects or treat a subject cells against oxidative stress.

15. The composition of claim 14, wherein the cells are isolated cells.

16.-18. (canceled)

19. A method for protecting and/or treating a subject against oxidative stress comprising administering an effective amount of the composition of claim 1 to said subject.

20. The method of claim 19, wherein said composition is administered to isolated cells of said subject.

21. The method of claim 19, wherein the subject is a human or an animal.
